(19) United States
(12) Patent Application Publication
(10) Pub. No.: US 2022/0366273 A1
(43) Pub. Date: Nov. 17, 2022

(54) PREDICTION OF RESIDUES OF PLANT PROTECTION AGENTS IN HARVESTED PRODUCTS

(71) Applicant: Bayer Aktiengesellschaft, Leverkusen (DE)

(72) Inventors: Kai WIRTZ, Düsseldorf (DE); Rolf Christian BECKER, Burscheid (DE); Linus GOERLITZ, Bergisch Gladbach (DE); Dirk SCHAEFER, Kerpen (DE); Fabian WEYSSER, Leverkusen (DE)

(73) Assignee: Bayer Aktiengesellschaft, Leverkusen (DE)

(21) Appl. No.: 17/633,568

(22) PCT Filed: Aug. 4, 2020
(86) PCT No.: PCT/EP2020/071849

§ 371 Date: Feb. 7, 2022

(30) Foreign Application Priority Data
Aug. 7, 2019 (EP) 19190498.6

(51) Int. Cl. G06N 5/02

(52) U.S. Cl. CPC G06N 5/022

(57) ABSTRACT

The present invention is concerned with the predicting of crop protection product residues in plants or parts of plants intended for human and/or animal consumption, preferably in fruit and/or vegetables. The present invention provides a method, a device, a system and a computer program product for prediction of crop protection product residues.

### CROSS REFERENCE TO RELATED APPLICATIONS

This application is a national stage application under 35 U.S.C § 371 of International Application No. PCT/EP2020/071849, filed Aug. 4, 2020, which claims the benefit of priority to European Application No. 19190498.6, filed Aug. 7, 2019.

### FIELD OF THE INVENTION

The present disclosure is concerned with the predicting of crop protection product residues in plants or parts of plants that are intended for human and/or animal consumption, preferably in fruit and/or vegetables. Specifically, the present disclosure provides a method, a device, a system and a computer program product for prediction of crop protection product residues.

### BACKGROUND OF THE INVENTION

Crop protection product residues are residues of active ingredients that were used for crop protection, i.e., were used during the production of plants, and are detectable in the end product.

The use of crop protection products is legally regulated in many countries. This is intended to ensure that crop protection products do not endanger animal and human health. Specific application regulations (for example, wait times or time intervals between the last application and harvest, amount to be applied, application limits) take the physical properties into account and hence influence the development and extent of residues.

For example, § 9 (1) of the German Lebensmittel-, Bedarfsgegenstände- und Futtermittelgesetzbuch (Lebensmittel- und Futtermittelgesetzbuch-LFGB) prohibits the commercial trading of food products if they contain crop protection products that exceed particular upper limits fixed for the individual substances.

The upper residue limits for crop protection products in conventional foods and animal feeds have been harmonized in all European member states since 1 Sep. 2008 by the entry into force of Directive (EC) No. 396/2005. However, there are no globally uniform limits.

Some store chains (traders) require their producers to produce plant-based foods with crop protection product residues (well) below the legal stipulations. There can be some variation between the requirements from different store chains.

Crop protection product residues can be determined and quantified in laboratories. There are numerous service providers that determine crop protection product residues to order in foods and animal feeds.

### SUMMARY OF THE INVENTION

It would be advantageous for a producer of plant-based foods and animal feeds to obtain, during the growing of the plants, information related to the amounts of crop protection product residues that are expected in the harvested products if the producer uses a defined crop protection product according to a defined application program. The producer would then be able to control the amount of residue of the crop protection product in the harvested product to a certain degree through changes in the crop protection product and/or changes in the application program. Moreover, it would be advantageous for such a producer to know which residue values are acceptable to which store chains to buy the producer's products and sell them on to end customers.

The present disclosure provides a method, a device, a system and a computer program product for prediction of crop protection product residues. In one or more embodiments, a device for predicting crop protection product residues comprises a processor configured to: receive and/or ascertain input information, wherein the input information comprises information relating to a crop plant being grown, information relating to the growing of the crop plant, information relating to a crop protection product that has been used or is planned to be used in the growing of the crop plant, and information relating to the application of the crop protection product; calculate, using the input information, an amount of a residue of the crop protection product in and/or on the parts of the crop plant intended for human and/or animal consumption at the time of harvesting of the crop plant; and output information relating to the amount of the residue.

In one or more embodiments, a method for predicting crop protection product residues comprises: receiving and/or ascertaining, by a processor, input information, wherein the input information relating to a crop plant being grown, information relating to the growing of the crop plant, information relating to a crop protection product that has been used or is planned to be used in the growing of the crop plant, and information relating to the application of the crop protection product; calculating, using a processor, an amount of a residue of the crop protection product in and/or on the parts of the crop plant intended for human and/or animal consumption, preferably at the time of harvesting; and outputting information relating to the amount of residue.

In one or more embodiments, a system for predicting crop protection product residues comprises two or more computer systems configured to: receive and/or ascertain, at a first computer system, input information comprising: information relating to a crop plant being grown, information relating to the growing of the crop plant, information relating to a crop protection product that has been used or is planned to be used in the growing of the crop plant, and information relating to the application of the crop protection product; transmit, by the first computer system, the input information to the second computer system via a network; receive, a the second computer system, the transmitted input information; calculate, by the second computer system, based on the input information, an amount of a residue of the crop protection product in and/or on the parts of the crop plant intended for human and/or animal consumption, preferably at the time of harvesting; transmit, by the second computer system, the amount of the residue to the first computer system via the network; receive, by the first computer system, the transmitted amount of the residue; output, by the first computer system, the amount of the residue.

In one or more embodiments, the present disclosure provides a non-transitory computer readable storage medium storing one or more programs for predicting crop protection product residue, wherein the programs are for execution by one or more processors of an electronic device and, when executed by the device, cause the device to: receive and/or ascertain input information, wherein the input information comprises information relating to a crop plant being grown, information relating to the growing of the crop plant, information relating to a crop protection product that has been used or is planned to be used in the growing of the crop plant, and information relating to the application of the crop protection product; calculate, using the input information, an amount of a residue of the crop protection product in and/or on the parts of the crop plant intended for human and/or animal consumption at the time of harvesting of the crop plant; and output information relating to the amount of the residue.

In some embodiments, a second method for predicting crop protection product residue comprises: specifying a crop plant, specifying growing parameters for growing of the crop plant, specifying a crop protection product, specifying application parameters for application of the crop protection product, calculating an amount of a residue of the crop protection product in and/or on the parts of the crop plant intended for human and/or animal consumption at the time of harvesting, and displaying the amount of residue to a user.

In some embodiments, the second method further comprises: ascertaining a maximum amount of a residue of the crop protection product specified in the crop plant specified or parts thereof; comparing the calculated amount of the residue with the maximum amount ascertained; and displaying to the user the extent to which the amount of the residue calculated exceeds the maximum amount.

In some embodiments of the second method, the maximum amount is an upper limit for a residue of the crop protection product specified in the crop plant specified or parts thereof that has been officially approved for a country or a region.

In some embodiments of the second method, the maximum amount is an upper limit for a residue in the crop protection product specified or parts thereof that has been stipulated by a dealer for the crop protection product.

In some embodiments, the second method further comprises: displaying those countries/regions for which the amount of the residue calculated does not exceed any legally approved upper limit for the residue in the specified crop plant or parts thereof and/or displaying those traders for which the amount of the residue calculated does not exceed any stipulated upper limit for the residue in the specified crop plant or parts thereof.

In some embodiments of the second method, the crop plant is specified by stating the crop plant variety being grown, wherein the growth parameters are specified by stating the country or region in which the crop plant is being grown, wherein the application parameters are specified by stating the application rate of the crop protection product (to be) applied and the length of the period of time between the juncture of application and the juncture of harvesting.

In some embodiments of the second method, the amount of the residue of the crop protection product is calculated at least using the following pieces of information: crop plant being grown, country or region in which the crop plant is being grown, crop protection product that has been or is to be used, application rate of the crop protection product that has been or is to be used, and length of the period of time between the juncture of application and the juncture of harvesting.

In some embodiments, the amount of the residue of the crop protection product is calculated at least using the following pieces of information: crop plant being grown, crop protection product that has been or is to be used, application rate of the crop protection product that has been or is to be used, amount of biomass and/or fruit mass present in the crop plant being grown at the juncture of application of the crop protection product, and length of the period of time between the juncture of application and the juncture of harvesting.

In some embodiments, the amount of the residue of the crop protection product is calculated using a regression model.

In some embodiments, the amount of the residue of the crop protection product is calculated using a self-learning algorithm that has been trained by means of empirical data to predict the amount of the residue of the crop protection product using information, wherein the information includes: crop plant being grown, country or region in which the crop plant is being grown or amount of biomass and/or fruit mass present in the crop plant being grown at the juncture of application of the crop protection product, crop protection product that has been or is to be used, application rate of the crop protection product that has been or is to be used, and length of the period of time between the juncture of application and the juncture of harvesting.

In some embodiments, the amount of the residue is displayed to a user as a proportion of a maximum amount stipulated by an official body and/or a trader.

In some embodiments, the second method further comprises: specifying variables relating to the crop plant being grown, to the growing parameters, to the crop protection products and/or to the application parameters that are variable and/or variables that are invariable, varying the variable variables in an automated manner and ascertaining those values of the variable variables for which the amount of the residue of the crop protection product reaches a minimum, and displaying the values of the variable variables at which the amount of the residue of the crop protection product reaches a minimum and displaying the minimum to the user.

In some embodiments, a device for predicting crop protection product residue comprises an input unit, a control and calculation unit and an output unit, wherein the control and calculation unit is configured to trigger the input unit to receive and/or ascertain the following information: crop plant being grown, growing parameters relating to the growing of the crop plant, crop protection product (to be) used, and application parameters relating to application of the crop protection product, wherein the control and calculation unit is configured to calculate an amount of a residue of the crop protection product in and/or on the parts of the crop plant intended for human and/or animal consumption at the time of harvesting, and wherein the control and calculation unit is configured to trigger the output unit to output the amount of the residue to a user.

In some embodiments, a system for predicting crop protection product residue comprises: a first computer system comprising an input unit, a first control and calculation unit, a first transmitter and receiver unit and an output unit, a second computer system comprising a second control and calculation unit and a second transmitter and receiver unit, wherein the first control and calculation unit is configured to trigger the input unit to receive and/or ascertain the following input information: crop plant being grown, growing parameters relating to the growing of the crop plant, crop protection product (to be) used, and application parameters relating to application of the crop protection product, wherein the first control and calculation unit is configured to trigger the first transmitter and receiver unit to transmit the input information via a network to the second computer system, wherein the second control and calculation unit is configured to trigger the second transmitter and receiver unit to receive the input information via the network, wherein the second control and calculation unit is configured to calculate an amount of a residue of the crop protection product in and/or on the parts of the crop plant intended for human and/or animal consumption at the time of harvesting, on the basis of the input information, wherein the second control and calculation unit is configured to trigger the second transmitter and receiver unit to transmit the amount of the residue via the network to the first computer system, wherein the first control and calculation unit is configured to trigger the first transmitter and receiver unit to receive the amount of the residue via the network, and wherein the first control and calculation unit is configured to trigger the output unit to output the amount of the residue to a user.

In some embodiments, a computer program product is provided comprising a data carrier and program code which is stored on the data carrier and which triggers a computer system, in the memory of which the program code has been loaded, to execute the following steps: receiving and/or ascertaining the following information: crop plant being grown, growing parameters relating to the growing of the crop plant, crop protection product (to be) used, and application parameters relating to application of the crop protection product; calculating an amount of a residue of the crop protection product in and/or on the parts of the crop plant intended for human and/or animal consumption at the time of harvesting; and displaying the amount of residue to a user.

### BRIEF DESCRIPTION OF THE DRAWINGS

The invention will now be described, by way of example only, with reference to the accompanying drawings, in which:

FIG. 1 shows an exemplary device for predicting crop protection product residue according to one or more embodiments of the disclosure.

FIG. 2 shows an exemplary device for predicting crop protection product residue according to one or more embodiments of the disclosure.

FIG. 3 shows an exemplary system for predicting crop protection product residue according to one or more embodiments of the disclosure.

FIG. 4 shows an exemplary method for predicting crop protection product residue according to one or more embodiments of the disclosure.

FIG. 5 shows an exemplary method for predicting crop protection product residue according to one or more embodiments of the disclosure.

FIG. 6 shows an exemplary method for predicting crop protection product residue according to one or more embodiments of the disclosure.

FIG. 7 shows displays of the computer program product of the invention on a screen of the device of the invention or of the system of the invention.

FIG. 8 shows an exemplary input mask via which a user can log in with their username and a password.

FIG. 9 shows a first display for the process for specifying a new field according to one or more embodiments of the disclosure.

FIG. 10 shows an exemplary first display for the process for specifying a new field with an exemplary first point in the field specified.

FIGS. 11-12 show exemplary points placed by a user according to one or more embodiments of the disclosure.

FIG. 13 shows an exemplary field and the calculated size of the field according to one or more embodiments of the disclosure.

FIG. 14 shows another display of the computer program of the invention according to one or more embodiments of the disclosure.

FIG. 15 shows another display of the computer program of the invention according to one or more embodiments of the disclosure.

FIG. 16 shows an exemplary display for creation of a growing plan for a crop plant according to one or more embodiments of the disclosure.

FIG. 17 shows another exemplary display of the computer program of the invention.

FIG. 18 shows an exemplary overview of markets ordered by country according to one or more embodiments of the disclosure.

FIG. 19 shows an exemplary display with a number of input fields for specifying an application program for at least one crop protection product according to one or more embodiments of the disclosure.

FIG. 20 shows an exemplary display with results of a prediction according to one or more embodiments of the disclosure.

FIG. 21 shows, in the form of a graph, an exemplary comparison between measured amounts of residue and predicted amounts of residue according to one or more embodiments of the disclosure.

### DETAILED DESCRIPTION

The invention will be more particularly elucidated hereinafter without distinguishing between the subjects of the invention (method, device, system, computer program product). Instead, the elucidations that follow are intended to apply analogously to all the subjects of the invention, irrespective of the context (method, device, system, computer program product) in which they occur.

If steps are stated in an order in the present description or in the claims, this does not necessarily mean that the invention is restricted to the stated order. Instead, it is conceivable that the steps can also be executed in a different order or else in parallel to one another, unless one step builds upon another step, which necessarily means that the step building upon the other is executed subsequently (but this will be clear in the individual case). The stated orders are thus preferred embodiments.

The present invention is executed with the aid of one or more computer systems.

A “computer system” is a system for electronic data processing that processes data by means of programmable computation rules. Such a system typically comprises a “computer”, i.e. unit comprising a processor for the execution of logic operations, and also peripherals.

In computer technology, “peripherals” refers to all devices that are connected to the computer and are used for control of the computer and/or as input and output devices. Examples thereof are monitor (display), printer, scanner, mouse, keyboard, drives, camera, microphone, speakers, etc. Internal ports and expansion cards are also regarded as peripherals in computer technology.

Modern computer systems are frequently divided into desktop PCs, portable PCs, laptops, notebooks, netbooks and tablet PCs, and what are called handhelds (e.g. smartphones); all these devices can be utilized for execution of the invention.

Inputs into the computer are achieved via input means such as, for example, a keyboard, a mouse, a microphone, a network connection, an external data memory and/or the like. “Input” is also to be understood as meaning the selection of an entry from a virtual menu or from a virtual list or clicking on a checkbox and the like. Outputs are usually achieved via a screen (monitor), on a printer, via loudspeakers and/or by storage on a data memory.

In some embodiments, the device of the present disclosure may be executed in the form of such a computer system. In some embodiments, the system of the invention may comprise two or more computer systems of this kind.

In some embodiments, the present disclosure serves to predict the amount of residue of a crop protection product in and/or on the parts of a crop plant intended for human and animal consumption, preferably at the time of harvesting.

The prediction may be made for a specific crop plant. The term “crop plant” may be understood to mean a plant which is specifically grown as a useful plant by human intervention. Parts of the crop plant being grown may be suitable for human and/or animal consumption. In some embodiments, the crop plant is a fruit plant or a vegetable plant.

In one or more embodiments, the prediction is made based on information that (further) specifies the crop plant, the crop protection product, and growing conditions. These pieces of information may also be referred to as “input information” in this description. The term “input information” shall not be understood here to mean that this information is all input into the computer system of the invention by a user. Instead, some of the input information may also be ascertained by the computer system of the invention (on the basis of information which has been input) (as elucidated in detail hereinafter). In this respect, the basis of the term “input” is more that the input information is included as input in the calculation of an amount of a residue of a crop protection product.

Also used in the present description is the term “specifying”. According to the manner in which an article is “specified”, the term “specifying” can have the meanings of “inputting”, “ascertaining”, “selecting”, “preselecting”, “calculating” and/or “deriving”.

In a first step, the crop plant may be specified. In some embodiments, this is preferably effected in that the user inputs the name of the crop plant or the name of the variety of the crop plant or a code for the crop plant/crop plant variety (for example according to the International Code of Nomenclature for Cultivated Plants, ICNCP for short) into a computer system, or selects the corresponding information relating to the crop plant from a list or menu, or selects a crop plant from a pictorial illustration of the crop plant (for example a photograph or a graphic).

It is also conceivable that the crop plant, or the vessel in which the crop plant is present, or a bed in which the crop plant is being grown, or a catalogue in which the crop plant is listed, or a package for the crop plant or for seed for growing of the crop plant, may be provided with a machine-readable code that provides information relating to the crop plant. In such a case, the specification of the crop plant may involve reading of the machine-readable code with a suitable reading device and transmission of the information relating to the crop plant that has been read by means of the reading device to the computer system of the invention. Such a machine-readable code may, for example, be an optoelectronically readable code (e.g. barcode, 2D code (e.g. DataMatrix or QR code)) or a code stored electronically in a semiconductor memory (e.g. an RFID chip).

It is also conceivable that the computer program product of the invention may be intended solely for a single crop plant, i.e. can only calculate/predict the amount of a residue of a crop protection product for a single crop plant or crop plant variety. In such a case, the step of “specifying a crop plant” involves a user selecting the corresponding computer program product to calculate amounts of residue of one or more crop protection products in the crop plant for which the computer program is intended.

In some embodiments, the crop plant is preferably selected from the following list: strawberry, tomato, cucumber, bell pepper, radish, kohlrabi, carrot, celery, fennel, parsnip, peas, beans, asparagus, spinach, chard, artichoke, black salsify, butterhead lettuce, crisphead lettuce, leaf lettuce, endive, chicory, aubergine, pumpkin, melon, courgette, lamb's lettuce, sugarbeet, rhubarb, white cabbage, red cabbage, kale, Brussels sprouts, cauliflower, broccoli, raspberry, blackberry, elderberry, cherry, apple, pear, grape, plum.

In a further step, information relating to the growing of the crop plant (also referred to in this description as growing parameters or growing conditions) may be specified.

In some embodiments, the growing parameters may specify where and/or under what conditions the crop plant is being grown. It may be possible to specify, for example, whether the crop plant is being grown outdoors, in a film tunnel (as is common practice for strawberries for example), in a greenhouse or the like. By virtue of the substantial lack of weather effects (e.g. rain), amounts of residue in plants that are cultivated in a greenhouse may be higher than in the case of cultivation outdoors.

In some embodiments, it may be possible to specify whether artificial irrigation is being effected; if appropriate, the irrigation volumes may be specified.

Especially where a greenhouse is involved, the user may preferably be able to specify the conditions that exist in the greenhouse, for example the temperature (air, soil), humidity (air, soil) and carbon dioxide content in the air, for example in the form of a progression over time during the day/night, maximum value, minimum value, mean value (e.g. arithmetic mean (average)), variance, cumulative temperatures, cumulative radiations and/or the like.

The abovementioned conditions (temperature, humidity, carbon dioxide content, amounts of radiation) can of course also be specified for crop plants that are being grown outdoors or in a film tunnel. In some embodiments, the weather conditions during the vegetation period of the crop plant are preferably recorded. It is possible here to detect the following values: air temperature, air humidity, air pressure, wind speed, type of precipitation, amount of precipitation, insolation and the like. In some embodiments, it may be possible here, for example, to detect daily maximum and daily mean values (e.g. arithmetic means). The weather conditions may on the one hand have an effect on the degradation characteristics of an active ingredient, and on the other hand determine the growth characteristics of the crop plants.

In some embodiments, the geographic position of the field in which the crop plant is being grown may preferably be specified. Using the geographic position of the field, it may then be possible to read out values for the conditions mentioned that existed during the growth period (current values) or typically exist (historical mean values), for example from databases.

The term “field” may be understood to mean a spatially delimitable region of the surface of the earth under agricultural cultivation, in that such a field is planted with crop plants that are supplied with nutrients and harvested.

In some embodiments, the specification of the geographic position of the field may involve, for example, specifying that country in which the field is located. It is also conceivable that a region in which the field is located may be specified. Such a region may, for example, be a region with a defined climate that differs from the climate of adjacent regions. A region may be a growing area for a specific crop plant (for definition of a growing area see, for example, Journal für Kulturpflanzen, 61 (7), p. 247-253, 2009, ISSN 0027-7479). A region may be a biome (for definition of equivalent German term Boden-Klima-Raum see, for example, Nachrichtenbl. Deut. Pflanzenschutzd., 59(7), p. 155-161, 2007, ISSN 0027-7479).

In some embodiments, the specification of the geographic position of a field may also involve giving the geographic coordinates (geocoordinates) of at least one point that lies within the field or lies at the edge of the field. Many fields may have the shape of a polygon. For such a field, specification may comprise giving the geocoordinates of the vertices of the polygon. It is conceivable that a user may draw the field boundaries on a virtual map on a display of the computer system of the invention by a finger or an input device (for example with a mouse) to specify the geographic position of a field.

It is conceivable that information relating to growing (e.g. typical growth conditions) for one or more crop plants may already be recorded on a data medium. It is conceivable that the device or system of the present disclosure, after the specifying of a crop plant, may read the customary growth conditions for the crop plant from the data medium and uses the values read as the basis for further calculation of the amount of residue. It is also conceivable that the growth conditions may be set by specifying the position; for defined countries and/or regions, it may be possible to store customary growth conditions on the data medium that can be made the basis for further calculation. It is also conceivable that the computer program product of the invention may be intended only for a single crop plant which is grown under predefined growth conditions. In such a case, the step of “specifying the growth conditions” may involve a user selecting the corresponding computer program product.

In some embodiments, in a next step, at least one crop protection product which is being used, i.e. applied, in the course of growth of the specified crop plant may be specified.

In some embodiments, the term “crop protection product” may be understood to mean a product that serves to protect plants or plant products from harmful organisms or prevent the effect thereof, to destroy unwanted plants or parts of plants, to inhibit unwanted growth of plants or prevent such growth, and/or to influence the life processes of plants as nutrients in some other way (e.g. growth regulators). Said growth regulators may serve, for example, to increase stability in cereals by shortening culm length (culm shorteners or, better, internode shorteners), improve the root development of seedlings, reduce the plant height by stunting in horticulture, or prevent the germination of potatoes. Growth regulators are typically phytohormones or their synthetic analogues. Examples of further crop protection products are herbicides, fungicides and other pesticides (e.g. insecticides, nematicides, molluscicides and the like).

In some embodiments, a crop protection product typically comprises one or more active ingredients. “Active ingredients” may refer to substances that have a specific effect in an organism and cause a specific reaction. An active ingredient may be a synthetically prepared (chemical) active ingredient or a (biological) active ingredient obtained from an organism. Combinations are also conceivable. A crop protection product may comprise a carrier for dilution of the one or more active ingredients. In addition, additives such as preservatives, buffers, dyes and the like may be conceivable. In some embodiments, a crop protection product may be in solid, liquid or gaseous form.

In some embodiments, a “harmful organism” may be understood to mean an organism that can appear in the course of growing crop plants and damage the crop plant, adversely affect the harvest of the crop plant or compete for natural resources with the crop plant. Examples of such harmful organisms are broadleaved weeds, weed grasses, animal pests, for example beetles, caterpillars and worms, fungi and pathogens (e.g. bacteria and viruses). Even though viruses are not organisms from a biological point of view, they shall nevertheless be covered by the term “harmful organism” in this description.

The term “broadleaved weed” (plural term: broadleaved weeds) may be understood to mean spontaneously accompanying vegetation plants in crops of crop plants, grassland or gardens that are not being specifically grown there and develop, for example, from the seed potential of the soil or are blown in. The term is not limited to broadleaved plants in the actual sense, but also includes grasses, ferns, mosses or woody plants. In the field of crop protection, the term “weed grass” (plural term: weed grasses) is frequently also utilized in order to illustrate a delimitation from the broadleaved plants. In the present text, the term “broadleaved weed” is used as an umbrella term that is to include weed grass, unless reference is being made to specific broadleaved weeds or weed grasses.

In some embodiments, the term “control” may be understood to mean prevention of infestation of a field/a crop plant or a portion thereof with one or more harmful organisms and/or prevention of the spread of one or more harmful organisms and/or reduction in the amount of harmful organisms present.

The specification of a crop protection product may be important for two reasons: firstly, the specification of the crop plant also specifies the substance(s) for which the amount(s) of residue is/are to be predicted in accordance with the invention. The substance for which an amount of residue is to be predicted may typically be the active ingredient in the crop protection product and/or a degradation product of the active ingredient that (likewise) exerts a biological effect in an organism. Secondly, degradation characteristics (and hence also the amount of residue) may be determined to a crucial degree by the chemical structure of the active ingredient and, if appropriate, the formulation thereof in the crop protection product. For many active ingredients, there are available models for degradation characteristics (see, for example, Environ. Sci. Technol. 2019, 53, 5838-5847; Soulas, G. & Lagacherie, B. Biol Fertil Soils (2001) 33: 551. https://doi.org/10.1007/s003740100363; Beulke, S. & Brown, C. D. Biol Fertil Soils (2001) 33: 558. https://doi.org/10.1007/s003740100364; Pagel, Holge, et al., Biogeochemistry, vol. 117, 2014, pp. 185-204, www.jstor.org/stable/24716853; https://www.epa.gov/pesticide-science-and-assessing-pesticide-risks/guidance-calculate-representative-half-life-values). Often, there may be exponential degradation; in other words, the amount of active ingredient deployed may decrease exponentially with time. In addition, the physicochemical properties of the active ingredient determine the extent to which the active ingredient is transported via the distribution pathways of the plants into edible components.

In some embodiments, a crop protection product can be specified, for example, based on the product name of the crop protection product or another name or the chemical formula for an active ingredient present in the crop protection product.

In one or more examples, a crop protection product may be selected from a list using a name and/or a pictorial representation (e.g. a photograph of the product).

In one or more examples, the packaging of the crop protection product may include a machine-readable code that provides information relating to the crop protection product and that can be read out by a suitable reading device. The machine-readable code may, as already described above, be an optoelectronically readable code and/or a code stored electronically in a semiconductor memory (for example of an RFID tag).

In some embodiments, multiple crop protection products that are/are to be applied simultaneously or at different times may be specified.

In one or more examples, the computer program product of the invention is intended solely for a single crop protection product, i.e. can only calculate/predict the amount of a residue of a specific crop protection product. In such a case, the step of “specifying a crop protection product” may involve a user selecting the corresponding computer program product to calculate amounts of residue of that crop protection product in a crop plant for which the computer program is intended.

In some embodiments, the device of the invention and the system of the invention may be configured such that they make a preselection in respect of the at least one crop protection product on the basis of the information relating to the crop plant being grown and/or on the basis of the information relating to the geographic position of the field and/or on the basis of the growing conditions. The term “preselection” in connection with a crop protection product means that the device/system selects from a list of crop protection products, and displays to the user, those crop protection products that are typically used for the crop plant specified and/or are typically used for control of harmful organisms that can occur in the case of the crop plant specified, and/or selects those crop protection products that are effective against harmful organisms that can appear under the conditions that exist at the geographic position of the specified field or in the greenhouse. A user may then be able to select one (or more) from the preselected crop protection products for which a calculation of the amount of residue is to be made. In one or more examples, the preselection finds only one suitable crop protection product, which can then be displayed to the user.

The list of preselected crop protection products may vary according to the specified geographic position for the field. The device of the invention and the system of the invention may preferably be configured such that they preselect and display only those crop protection products that have been officially approved for deployment in the country in which the respective field is located. Corresponding information relating to official approvals may be stored in one or more databases.

In some embodiments, in a further step, the application of the crop protection product may be preferably specified (in detail) using application parameters. The application parameters may include the type of treatment, the application rate and at least one juncture or at least one period of time at which/in which the crop protection product is (to be) applied in the amount specified. The earlier a treatment is effected in the vegetation period of the crop plant, the smaller the amount of residues typically is.

In some embodiments, it is conceivable that crop protection product may be applied repeatedly over a vegetation period (at different junctures or in different periods of time). In the case of multiple application, it may be possible to specify the number of applications and the time interval between the applications. The application rates may be the same or may vary in the case of multiple deployment. There may also be variation in the nature of treatment and/or the crop protection product used in each case. A factor of major importance may be the period of time between the last application of a crop protection product and the harvest; in general, crop protection product residues are degraded over time; i.e., the longer ago the juncture of the application, the smaller the amount of residue.

In the specification of the type of treatment, for example, it may be possible to state whether the treatment is of the seed or of the plant at a defined stage of development. Treatments applied directly on the harvested material normally lead to higher amounts of residue than, for example, seed treatments applied prior to sowing. In addition, it may be possible to state which parts of the plant are treated (for example leaves, fruits and/or roots) or whether soil treatment takes place.

The “application rate” may be the amount of a crop protection product needed for control of harmful organisms, which may be typically reported per unit area (on deployment to the field), per unit space (for example in a greenhouse) or per unit of amount of seed (for example in the case of seed dressing). In some embodiments, the amount can be reported in the form of weight (e.g. kilograms) or in the form of volume (e.g. liters). The amounts may be based on the total amount of the crop protection product or on the active ingredient present in the crop protection product. It tends also to be the case that the greater the amount of active ingredient deployed to the area, the higher the amount of residue in/on the harvested material.

In some embodiments, if water is used for dilution of a crop protection product, it may also be possible to specify the amount of water used.

In some embodiments, the application parameters can be input by a user into the device of the invention or the system of the invention. In addition, the application parameters may be wholly or partly ascertained by the device of the invention or the system of the invention using the information already input in relation to the crop plant, to the geographic position of the field, to the growth conditions and/or to the crop protection product.

It is conceivable, for example, that information related to when and in what amounts the crop protection product should be applied may be provided by the manufacturer or distributor of the crop protection product. This information may be stored in an internal or external data medium that can be accessed by the device of the invention/the system of the invention. The device/system may thus be configured to generate a suggestion on the basis of the information available as to when the crop protection product should be applied and in what amounts.

Junctures/periods of time at which/in which crop protection products are applied may, for example, (also) be guided by the stage of development of the crop plant. The stage of development of a plant may be stated, for example, in the form of what is called the BBCH code. Officially, the BBCH abbreviation denotes the Biologische Bundesanstalt, Bundessortenamt and CHemische Industrie [Federal Biological Institute for Agriculture and Forestry, Federal Office for Crop Plant Varieties, Chemical Industry]. The extended BBCH scale for uniform coding of the phenological stages of growth of mono- and dicotyledonous plants is a partnership between the Biologische Bundesanstalt für Land- and Forstwirtschaft (BBA), the Bundessortenamt (BSA), the Industrieverband Agrar (IVA) [Agrochemical Industry Association] and the Institute of Vegetable and Ornamental Crops in Grossbeeren, Erfurt. It is conceivable that a user inputs the (extended) BBCH code for the crop plant being grown into the device/system. It is additionally conceivable that the device/system is configured to calculate the BBCH code itself. It is conceivable, for example, that the user has input the time of planting/sowing of the crop plant into the device/system, and the device/system calculates the BBCH code using the time of planting/sowing.

In some embodiments, the device/system of the invention may be configured to calculate the stage of growth of the crop plant with the aid of a plant growth model. Such a plant growth model may, for example, be a mathematical model that describes the growth of a plant depending on intrinsic (genetic) and extrinsic (environmental) factors. An overview of the creation of plant growth models is given, for example, by the books i) “Mathematische Modellbildung and Simulation” [Mathematical Model Formation and Simulation] by Marco Gunther and Kai Velten, published by Wiley-VCH Verlag in October 2014 (ISBN: 978-3-527-41217-4), and ii) “Working with Dynamic Crop Models” by Daniel Wallach, David Makowski, James W. Jones and Francois Brun, published in 2014 in Academic Press (Elsevier), USA.

Plant growth models exist for a multitude of crop plants. The plant growth model typically simulates the growth of a crop of crop plants over a defined period of time. It may also be conceivable to use a model based on a single plant that simulates the flows of energy and matter in the individual organs of the plant. In one or more examples, mixed models are additionally usable.

The growth of a crop plant is determined not only by the genetic features of the plant but primarily also by the local weather conditions that exist over the lifetime of the plant (quantity and spectral distribution of the incident solar radiation, temperature profiles, amounts of precipitation, wind input), the condition of the soil and the nutrient supply.

Cultivation measures that have already been undertaken (in the past) and any infestation with harmful organisms that has occurred can also influence crop growth and can be taken into account in the growth model.

The plant growth models are generally what are called dynamic process-based models (see “Working with Dynamic Crop Models” by Daniel Wallach, David Makowski, James W. Jones and Francois Brun, published in 2014 in Academic Press (Elsevier), USA), but may also be entirely or partly rule-based or statistical or data-assisted/empirical. The models are generally what are called point models. The models here are generally calibrated such that the output reflects the three-dimensional representation of the input. If the input is ascertained at a point in space or is interpolated or estimated for a point in space, it is generally assumed that the model output is applicable to the entire adjoining field. An application of so-called point models calibrated to the field level to further, generally larger scales is known (see, for example: H. Hoffmann et al.: Impact of spatial soil and climate input data aggregation on regional yield simulations. PLoS ONE 11(4): e0151782. doi:10.1371/journal.pone.0151782). Application of this so-called point model to multiple points within a field enables part-area-specific modelling here. However, spatial dependences are neglected here, for example in the groundwater budget. However, there also exist systems for space/time-explicit modelling. These take account of spatial dependences.

Examples of dynamic, process-based plant growth models are Apsim, Lintul, Epic, Hermes, Monica, STICS inter alia. A comparison of the models and corresponding literature relating to the models can be found, for example, in the following publication and references cited therein: H. Hoffmann et al.: Impact of spatial soil and climate input data aggregation on regional yield simulations. PLoS ONE 11(4): e0151782. doi:10.1371/journal.pone.0151782.

In some embodiments, the modelling of plant growth may include the following parameters:

(a) Weather: daily precipitation totals, radiation totals, daily minimum and maximum air temperatures and temperature close to the ground and soil temperature, wind speed, inter alia
(b) Soil: soil type, soil texture, soil character, field capacity, permanent wilting point, organic carbon, mineral nitrogen content, lodging density, van Genuchten parameters, inter alia
(c) Crop plant: species, variety, species-specific parameters, for example specific leaf area index, temperature totals, maximum root depth, inter alia.
(d) Cultivation measures: seed, sowing date, sowing density, sowing depth, fertilizer, amount of fertilizer, number of fertilizer dates, fertilizer date, soil conditioning, harvest residues, crop rotation, distance from the field of the same crop the year before, irrigation, inter alia.

With the aid of a plant growth model, it may also be possible to calculate the amount of biomass present at any juncture and/or the size of the leaf area and/or the amount of fruits (fruit mass) and/or the number of shoots present and/or the like. In some embodiments, a plant having more biomass and/or a greater leaf area requires a great amount of crop protection product than a plant having less biomass and/or a smaller leaf area. It may be conceivable that the amount of biomass present—especially in the form of fruit—at the time of application of a crop protection product exerts an influence on the amount of residue. In a preferred embodiment of the present invention, therefore, the amount of biomass of the crop plant being grown that is present at any juncture (especially at the juncture of application of a crop protection product) is included in the calculation of an amount of residue.

It may also be possible to ascertain and/or predict the amount of biomass present and/or to optimise plant growth models using remote sensing data. “Remote sensing data” are digital information that has been obtained remotely, for example by satellites, from the surface of the earth In one or more examples, it may be possible to use aircraft (unmanned (drones) or manned) for recording of remote sensing data. The remote sensors generate digital images of regions of the surface of the earth from which information can be obtained about the vegetation that exists there and/or the environmental conditions that exist there (see, for example, M. S. Moran et al.: Opportunities and Limitations for Image-Based Remote Sensing in Precision Crop Management, Remote Sensing of Environment (1997) 61: 319-346). The data from these sensors are sourced via the interfaces made available by the provider and may include optical and electromagnetic (e.g. Synthetic Aperture Radar SAR) datasets at various stages of processing.

In some embodiments, it may also be possible to use sensors in the field in order to determine the stage of growth and/or the available biomass of the crop plants. The sensors may be in a stationary position in the field; it may be conceivable to equip agricultural machinery and/or robots that move through the field with appropriate sensors.

Junctures/periods of time at which/in which crop protection products are applied may be guided by the stages of development and/or the spread of harmful organisms. Prediction models are also available for the development and/or spread of harmful organisms (see, for example: WO2017/222722A1, WO2018/058821A1, US20020016676, US20180018414A1, WO2018/099220A1). With the aid of such models, it may be possible to predict times at which there will be a high risk of infestation of the crop plants with harmful organisms. Control of the harmful organisms with a crop protection product may preferably be undertaken when the (calculated) risk of infestation is particularly high (for example surpasses a defined threshold). The models for prediction of infestation with a harmful organism also typically use weather data and historical data.

The device and system of the present disclosure may be configured to calculate, based on the information available (input information, information derived from the input information and/or information read out from a data medium or multiple data media using the input information), the amount of at least one residue of a crop protection product in and/or on the crop plant or in and/or on part of the crop plant, preferably at the time of harvesting. If there is still a significant reduction in the amount of residue after harvesting (for example during storage, as a result of washing and/or the like), the amount of residue can (also) be calculated for a different time from the time of harvesting and/or for a condition after a specific treatment (for example washing, exposure to electromagnetic radiation, heat or cold treatment and/or the like).

In some embodiments, the amount of the at least one residue can be calculated, for example, on the basis of empirically ascertained data relating to the distribution of crop protection products in parts of the crop plant and to the degradation of crop protection products.

In some embodiments, the amount of the at least one residue of a crop protection product may be calculated with the aid of a regression model on the basis of empirical data. The regression model may describe the correlation between the input information (e.g. geographical position of the field, crop plant, crop protection product and application parameters) and at least one output value (amount of residue of the crop protection product, preferably at the time of harvesting).

In one or more embodiments, the regression model can be created by means of machine learning. Preference is given to creating such a model by means of monitored learning. This may involve training the regression model to make a prediction for at least one output datum on the basis of input data. Training and validation of the model can be accomplished using empirical data. This means that, for a multitude of crop plants, growth conditions, crop protection products and application conditions, an empirical determination is made of the amounts of crop protection product residue that arise at the time of harvesting, for example. These empirically obtained data can then be used to create a corresponding regression model.

In the study of such empirical data, it has been found that, for a multitude of crop plants and crop protection products, it may be possible to predict the amounts of crop protection product residue using a few pieces of input information. This may require at least the following information (as input information, for example for the regression model): crop plant, growth conditions or a parameter that correlates with the growth conditions, crop protection product, application rate of the crop protection product and period of time between the application of the crop protection product and the harvesting time. An example of a parameter that correlates with the growth conditions is the amount of biomass present at the time of application, which, as described above, can be ascertained empirically by means of sensors and/or calculated by calculation with the aid of a plant growth model and/or specified by the user (for example after inspection). In a particularly preferred embodiment, the amount of residue may be predicted based on the following information (which may also be used for the training of the corresponding regression model): crop plant grown, land/or region in which the crop plant is growing or amount of biomass and/or fruit mass present in the crop plant grown at the time of application of the crop protection product (or a parameter that correlates therewith, for example the diameter of a plant or a fruit), crop protection product used or to be used, application rate of the crop protection product used or to be used, and length of the period of time between the juncture of application and the juncture of harvesting. It may be particularly advantageous when not only the input information mentioned but also one or more of the following further pieces of input information relating to weather conditions during the growth phase are included in the calculation: insolation (for example in the form of hours of sunshine), humidity and/or temperature.

Details of the creation of a regression model are described in the prior art (see, for example, Norman Matloff: Statistical Regression and Classification—From Linear Models to Machine Learning, Texts in Statistical Science, CRC Press 2017, ISBN 978-1-4987-1091-6; Pratap Dangeti, Statistics for Machine Learning, Packt Publishing 2017, ISBN 978-1-78829-575-8).

The regression model may preferably be a non-linear regression model (see, for example: Christian Ritz, Jens Carl Streibig: Nonlinear Regression with R, Springer Science & Business Media, 2008, ISBN 9780387096162; R. Russell Rhinehart: Nonlinear Regression Modeling for Engineering Applications: Modeling, Model Validation, and Enabling Design of Experiments, Wiley-ASME Press Series, John Wiley & Sons, 2016, ISBN 9781118597965; Hossein Riazoshams et al.: Robust Nonlinear Regression: with Applications using R, John Wiley & Sons, 2018, ISBN 9781118738061).

In some embodiments, the amount of residue may be calculated with the aid of a non-linear regression model based on the following input information:
crop plants being grown
crop protection product used
number of applications of the crop protection product and amounts applied
period of time between the last application and the harvesting time
average air and/or soil temperature on the respective days of application
average air humidity on the respective days of application
accumulated radiation energy (insolation or artificial lighting) on the respective days of application
average diameter and/or average height of the crop plants grown on the respective application days.

The amount of the residue ascertained can be output to a user. The output may take the form, for example, of text and/or numbers and/or graphics on a monitor (screen) and/or printer of the device/system of the invention.

In some embodiments, the amount of residue ascertained may be compared with a maximum amount or with multiple maximum amounts. The at least one maximum amount is, for example, the legally approved upper limit of a residue in a crop plant for the respective crop protection product.

The at least one maximum amount may also be an upper limit of a residue in a crop plant required by a trader for the respective crop protection product. A “trader” in the context of the present invention is a natural or legal person that buys crop plants or parts of crop plants from a producer and sells them on (for example to an end customer (consumer)).

The at least one maximum amount may also be an amount defined by a user. The at least one maximum amount may be stored in a data medium or multiple data media that can be accessed by the device of the invention and the system of the invention.

In some embodiments, the extent to which the amount of residue ascertained is above or below one or more maximum amounts may be displayed to the user The user may specify a country or region, and whether and/or the extent to which the amount of residue ascertained is above or below the upper limit approved for the specified country or region may be displayed to the user. It may be conceivable that the user can specify multiple countries/regions. In some embodiments, the user specifies a trader, and whether and/or the extent to which the amount of residue ascertained is above or below the upper limit approved by the specified trader is displayed to the user. In some embodiments, the user can specify multiple traders.

In some embodiments, the amount of the residue of the crop protection product ascertained may be reported in the form of a percent figure and/or in the form of a graphic representation of the percent figure, where the percent figure indicates the percentage of the amount of residue calculated of a maximum, preferably legally or officially permitted, amount of residue and/or a maximum amount of residue stipulated by a trader. In some embodiments, the user may be able to select a country/region or multiple countries/regions, and the size of the amount of residue calculated in relation to the maximum amount of residue stipulated in the country/region is displayed. In a preferred embodiment, the user is able to select one or more traders, and the size of the amount of residue calculated in relation to the maximum amount of residue stipulated by the trader is displayed to them.

In some embodiments, the calculated amount of residue may be based on the maximum residue limit/level (MRL) stipulated by an official body. The maximum residue limit is the maximum permissible residue concentration. In the EU, for example, the European Medicines Agency (EMA) is responsible for recommending maximum residue levels which, under its recommendation, are made legally valid standards for food safety by the European Commission.

The maximum residue level may normally be ascertained by repeated field trials (in the order of magnitude of 10) in which the harvest has been treated according to good agricultural practice (GAP), and an appropriate preharvest interval or an appropriate period of time has elapsed. For many pesticides, the upper residue limit is at their limit of detection (LOD). The limit of quantification (LOQ) is frequently used rather than the LOD. The rule of thumb is that the LOQ value is about twice the LOD value. For substances not included in any of the annexes of the EU standards, the standard maximum residue level normally applicable is 0.01 mg/kg (see e.g. https://ec.europa.eu/food/plant/pesticides/max_residue_levels_en). Instead of or as well as the MRL value, the amount of residue calculated can also be expressed in relation to other customary parameters, for example ARfD, ADI and/or TDI. Acute reference dose (ARfD) is an estimate of the amount of a substance in foods or drinking water that can be ingested by the consumer over a short period of time, normally during a mealtime or a day, without significant risk to health. Acceptable daily intake (ADI) refers to the dose of a substance that is considered to be medically benign in the case of lifelong daily ingestion. In the case of unwanted impurities, this is called tolerable daily intake (TDI).

In some embodiments, the device/system of the invention may be configured such that, at defined junctures or in the event of occurrence of defined events, the calculation of the amount of residue is updated to take account of a change in the growing conditions, in the crop protection products and/or in the application parameters. For example, a first calculation of the amount of residue may be based on a climate typical of the respective country or region in which the crop plant is being grown. Over the course of the vegetation period, the calculation is then adjusted to the actual weather conditions. In an analogous manner, the calculation can be updated on the basis of the applications of a crop protection product that have actually been implemented. In addition, an update can be made on the basis of sensor data (for example remote sensing data and/or field data), where the sensor data can give information, for example, as to the state of development of the crop plant and/or an expected harvest yield.

In some embodiments, the device/system of the invention may be configured such that the user can change the crop protection product specified and/or change the application parameters, after which the amount of a residue of the crop protection product is recalculated and displayed. This enables planning by the user. The user may be able to evaluate how a change in the crop protection product and/or in the application parameters affects the amount of residue.

In some embodiments, the device/system of the invention may be configured, in reaction to a user input for optimization of the amount of residue, to alter the crop protection product and/or the application parameters so as to result in a minimum amount of residue. In the case of such an optimization, it may be the case that the user can decide which parameters are variable and which are invariable. The device/system then alters the variable parameters until a minimum amount of residue is attained, and outputs the altered parameters and the (minimum) amount of residue calculated to the user. Methods of mathematical optimization can be found in the numerous textbooks on this topic (see, for example, Peter Gritzmann: Grundlagen der Mathematischen Optimierung [Fundamentals of Mathematical Optimization], Springer Spektrum 2013, ISBN: 978-3-528-07290-2).

The invention is more particularly elucidated below with reference to figures, without wishing to restrict the invention to the features and combinations of features that are shown in the figures.

FIG. 1 shows, by way of example and in schematic form, an embodiment of the device of the invention. The device (10) comprises an input unit (11), a control and calculation unit (12) and an output unit (13). A user can use the input unit (11) to input information and control commands into the device. The output unit (13) can output information to a user, preferably displayed on a monitor. The control and calculation unit (12) serves primarily to control the components of the device (10), to process the information that has been input and output and to perform calculations and logical operations.

The control and calculation unit (12) may be configured to
trigger the input unit (11) to receive the following information:
crop plant being grown
growing parameters relating to the growing of the crop plant
crop protection product (to be) used
application parameters relating to application of the crop protection product
use the information received to calculate an amount of a residue of the crop protection product in and/or on the parts of the crop plant intended for human and/or animal consumption, preferably at the time of harvesting, and
prompt the output unit (13) to output the amount of the residue to a user.

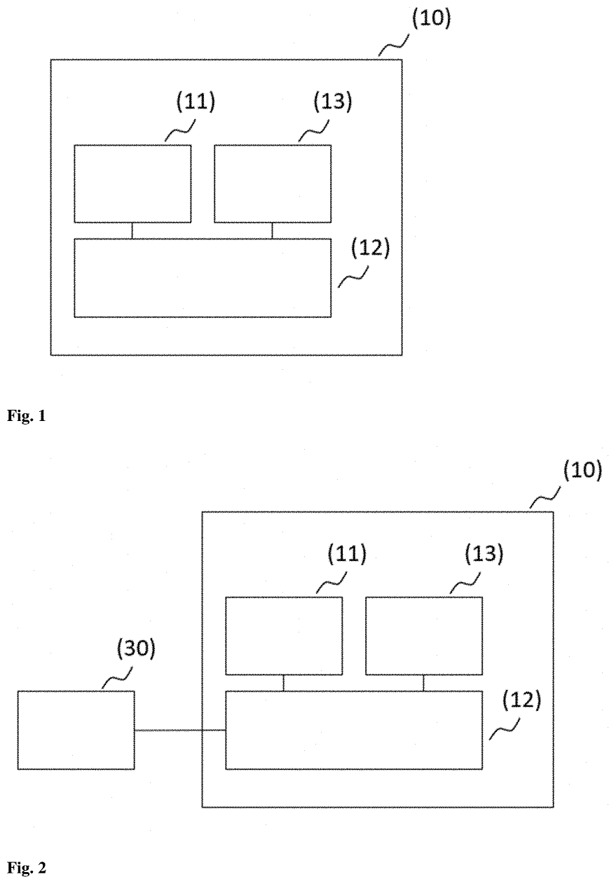

FIG. 2 shows, by way of example and in schematic form, a further embodiment of the device of the invention. As well as the input unit (11), the control and calculation unit (12) and the output unit (13) as described in relation to FIG. 1, the device (10) may be connected to a data medium (30), for example via a network. The data medium (30) can store, for example, information relating to crop plants (for example preferred growing conditions), to crop protection products (for example preferred application parameters), to the climate of a country or region, to the weather of a country or region, to the spread of harmful organisms in a country or region and the like. The control and calculation unit (12) may be configured to access the information stored on the data medium (30) and use it for calculation of the amount of the residue of a crop protection product. In addition, the control and calculation unit (12) may be configured to store information on the data medium (30). It is conceivable that the data medium (30) comprises multiple data media.

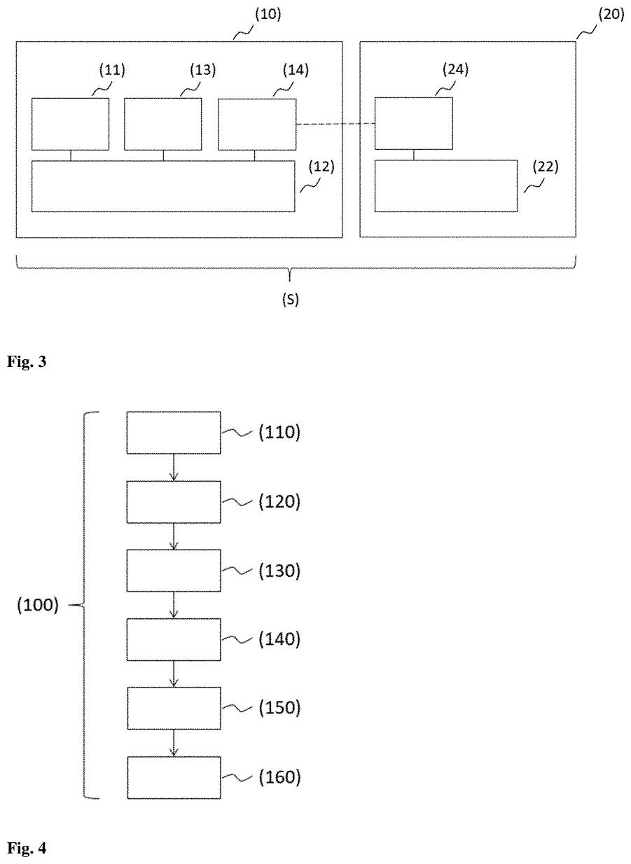

FIG. 3 shows, by way of example and in schematic form, an embodiment of the system of the invention. The system (S) comprises a first computer system (10) and a second computer system (20). The first computer system (10) preferably takes the form of a desktop, laptop or tablet computer or of a smartphone. The second computer system (20) preferably takes the form of a server. The first computer system (10) is operated by a user. The first computer system (10) serves as communication interface between the user and the system (S). The second computer system (20) serves to take on some functionalities that are executed by the control and calculation unit of the device of the invention. Reasons for the movement of functionalities to a second computer system may be:
calculations require a high computing power; these calculations are transferred to a server equipped with the corresponding computing power;
calculations should always be based on the newest versions of models and current data; these newest versions of models and current data are provided by means of a server.

Typically, there may be a multitude of first computer systems that are operated by different users, and only one second computer system or a small number (compared to the number of first computer systems) of secondary computer systems that provide resources (computing power, data, models) for the multitude of first computer systems via a network or multiple networks.

In some embodiments, the first computer system (10) comprises an input unit (11), a first control and calculation unit (12), an output unit (13) and a first transmitter and receiver unit (14). The second computer system (20) comprises a second control and calculation unit (22) and a second transmitter and receiver unit (24). The first computer system (10) and the second computer system (20) can exchange information via a network (represented by the dotted line) between the first transmitter and receiver unit (14) of the first computer system (10) and the second transmitter and receiver unit (24) of the second computer system (20). The network may comprise a mobile network, for example one based on the GSM, GPRS, 2G, 3G, LTE, 4G, 5G standard or another standard.

The first control and calculation unit (12) may be configured to receive the following input information from a user via the input unit (11):
crop plant being grown
growing parameters relating to the growing of the crop plant
crop protection product (to be) used
application parameters relating to application of the crop protection product.

The first control and calculation unit (12) may be configured to trigger the first transmitter and receiver unit (14) to transmit the input information via the network to the second computer system (20).

The second control and calculation unit (22) may be configured to trigger the second transmitter and receiver unit (24) to receive the input information via the network.

The second control and calculation unit (22) may be configured to calculate an amount of a residue of the crop protection product in and/or on the parts of the crop plant intended for human and/or animal consumption, preferably at the time of harvesting, on the basis of the input information.

The second control and calculation unit (22) may be configured to trigger the second transmitter and receiver unit (24) to transmit the amount of the residue via the network to the first computer system (10).

The first control and calculation unit (12) may be configured to trigger the first transmitter and receiver unit (14) to receive the amount of the residue via the network.

The first control and calculation unit (12) may be configured to trigger the output unit (13) to output the amount of the residue to the user.

The system (S) of the invention may comprise one data medium or multiple data media. Such a data medium can store information relating to crop plants (for example preferred growing conditions), to crop protection products (for example preferred application parameters), to the climate of a country or region, to the weather of a country or region, to the spread of harmful organisms in a country or region and the like. Such a data medium may be a constituent of the first computer system (10) or of the second computer system (20) and/or a separate unit that may be connected to the first computer system (10) and/or the second computer system (20) via a network.

FIG. 4 shows, by way of example and in schematic form, an embodiment of the method of the invention in the form of a flow diagram. The method (100) comprises the steps of:

(110) specifying a crop plant
(120) specifying growing parameters for growing of the crop plant
(130) specifying a crop protection product
(140) specifying application parameters for application of the crop protection product
(150) calculating an amount of a residue of the crop protection product in and/or on the parts of the crop plant intended for human and/or animal consumption, preferably at the time of harvesting
(160) displaying the amount of the residue to a user.

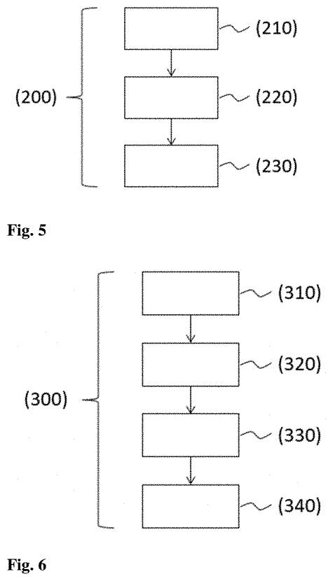

FIG. 5 shows, by way of example and in schematic form, the steps that are executed by a computer system on which the computer program product of the invention is installed in the form of a flow diagram. The steps (200) comprise:

(210) receiving the following information:
crop plant being grown
growing parameters relating to the growing of the crop plant
crop protection product (to be) used
application parameters relating to application of the crop protection product
   (220) calculating an amount of a residue of the crop protection product in and/or on the parts of the crop plant intended for human and/or animal consumption, preferably at the time of harvesting
   (230) displaying the amount of residue to a user.

FIG. 6 shows, by way of example and in schematic form, the steps that are executed by a computer system on which a preferred embodiment of the computer program product is installed in the form of a flow diagram. The steps (300) comprise:

(310) receiving the following input information from a user:
crop plant being grown
growing parameters relating to the growing of the crop plant
crop protection product (to be) used
application parameters relating to application of the crop protection product
   (320) transmitting the input information to a second computer system
   (330) receiving an amount of a residue of the crop protection product in and/or on the parts of the crop plant intended for human and/or animal consumption, preferably at the time of harvesting, from the second computer system
   (340) displaying the amount of residue to a user.

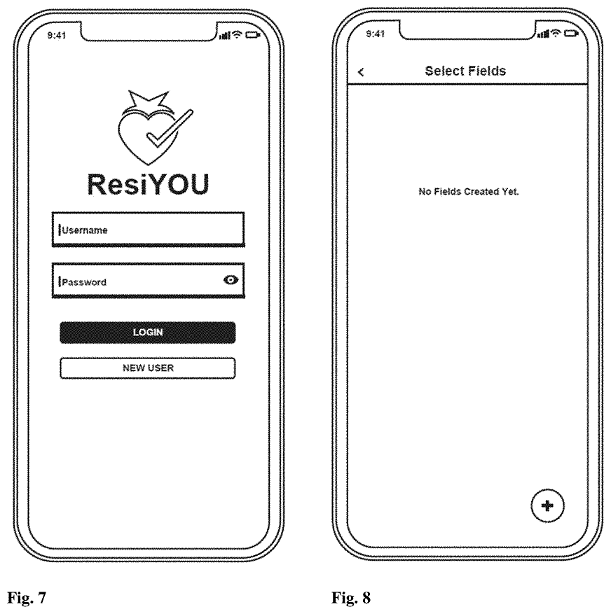

FIG. 7 to FIG. 20 show, by way of example, displays of the computer program product of the invention on a screen of the device of the invention or of the system of the invention.

FIG. 7 shows, by way of example, an input mask via which a user can log in with their username and a password.

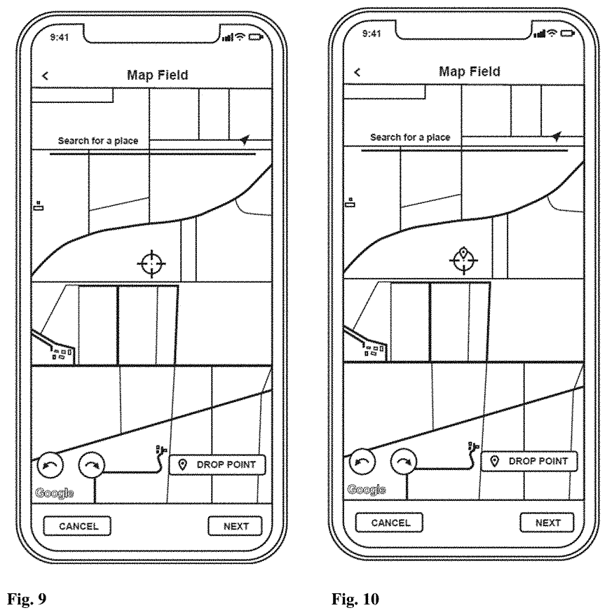

Once the user has logged in, they can be requested to specify a field in which the crop plant is being grown or is to be grown. FIG. 8 shows a display of the computer program product of the invention that indicates that no field has been specified yet (“No Fields Created Yet”). By clicking on the virtual button with the ⊕ symbol, a user can start a process for specifying a new field. The process is illustrated in the form of images in FIG. 9 to FIG. 13.

FIG. 9 shows a first display for the process for specifying a new field. It is possible to input the name of a location (e.g. country, place and/or street) into an input field (“Search for a place”). The background shows a detail of the surface of the earth in an aerial photograph or in the form of a map. On input of the name of a location, a detail of the surface of the earth which includes the location may be shown. By means of the finger movements known from the use of smartphones, the user is able to move the detail, enlarge the detail (zoom out) or reduce the size of the detail (zoom in).

In addition, by means of a virtual button (“drop point”), it may be possible to specify a point within the field or at the edge of the field. On actuation of the virtual button, such a point is put in the middle of the target. This is shown in FIG. 10. FIG. 10 shows the same display as FIG. 9, except that a first point in the field has now been specified.

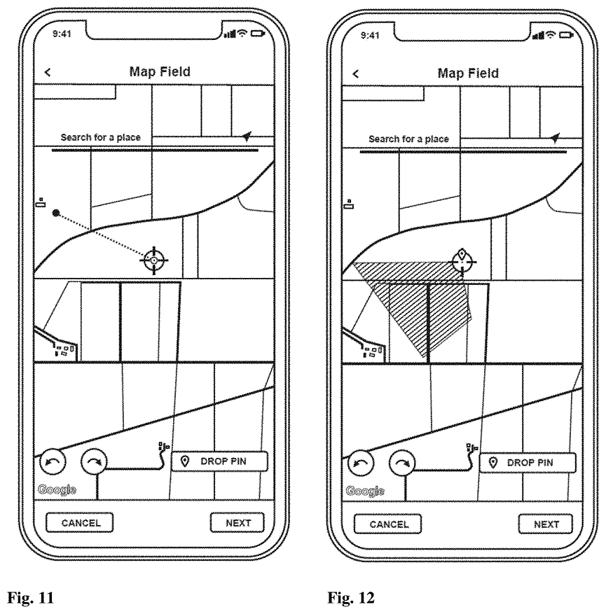

Proceeding from the point specified, a user can then place (fix) further points of the field. The points are joined to one another by straight lines. This is shown in FIG. 11 and FIG. 12.

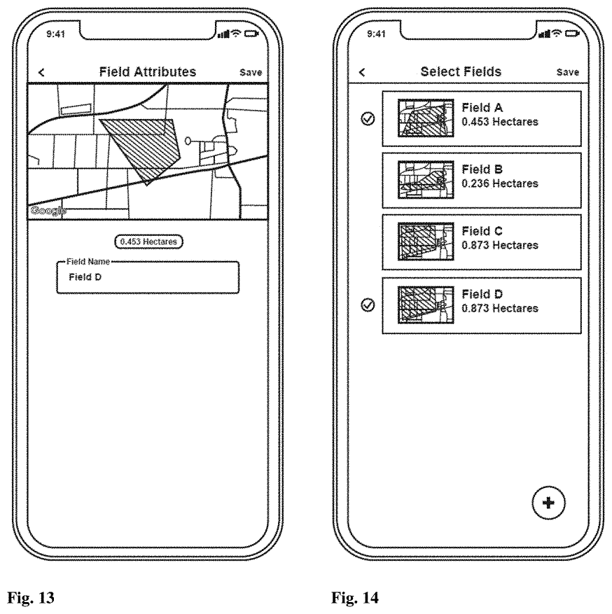

The computer program may be configured such that it automatically calculates the size for a field specified. FIG. 13 shows the field specified and the calculated size of the field (“0.453 Hectares”). The user can give a name to the field (“Field Name”); in the present case it is named “Field D”.

FIG. 14 shows a further display of the computer program of the invention. It is shown that four fields named “Field A”, “Field B”, “Field C”, “Field D” have been set out. The user can select those fields for which they would like to have a prediction of an amount of residue of a crop protection product. The virtual button with the ⊕ symbol indicates that further fields can be specified (set out in the computer program).

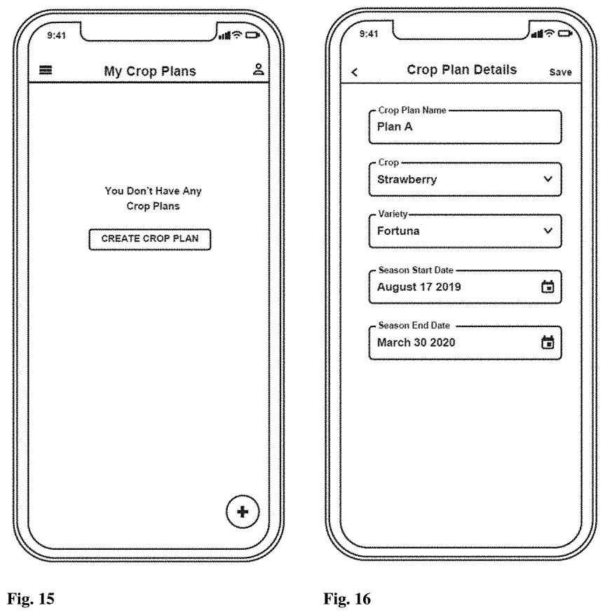

FIG. 15 shows a further display of the computer program of the invention. The computer program indicates to the user that no information has been specified to date as to the growing of a crop plant and the application of a crop protection product (“You Don't Have Any Crop Plans”). By actuating a virtual button (“Create Crop Plan”), the user may be able to start the process for specifying the corresponding information. The process is illustrated in the form of images in FIG. 16.

FIG. 16 shows a display for creation of a growing plan for a crop plant (“Crop Plant Details”). The display comprises a series of input fields. In one input field, the user can give the respective growing plan a name (“Crop Plan Name”). In the present case, the name input by the user was “Plan A”. In a further input field, the user can specify the crop plant (“Crop”). In the present case, this may be accomplished by selection of an entry in a list. In the present case, the user has selected strawberry as the crop plant. In a further input field, the user can specify the crop plant variety (“Variety”). In the present case, the user has selected Fortuna as the crop plant variety. In a further input field, the user can specify the start of the growing period (“Season Start Date”). This can be effected by entering a date and/or by selecting a day in a virtual calendar. In the present case, the user has specified 17 Aug. 2019 as the start of the growing period. In a further input field, the user can specify the end of the growing period (“Season End Date”). This can be effected by entering a date and/or by selecting a day in a virtual calendar. In the present case, the user has specified 30 Mar. 2020 as the end of the growing period.

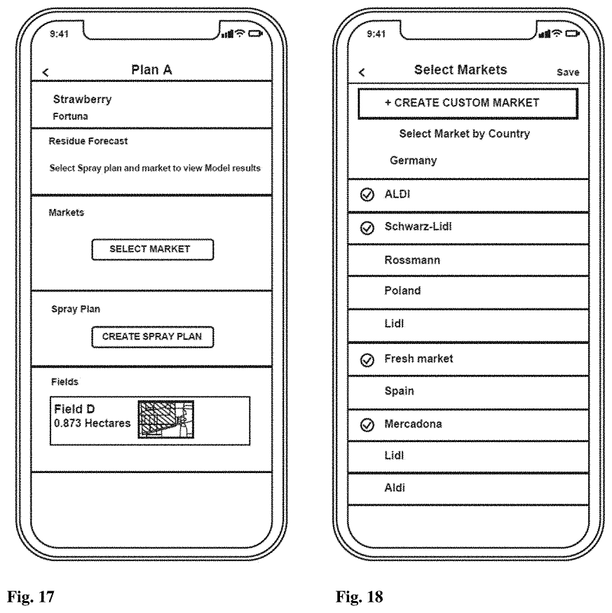

FIG. 17 shows a further display of the computer program of the invention. FIG. 17 indicates a growing plan named Plan A in an overview. The growing plan relates to the strawberry crop plant of the Fortuna variety. A field named “Field D” has been specified, on which the crop plant is (to be) grown. Before the computer program can calculate (predict) an amount of a residue of a crop protection product, it may be necessary to specify one or more markets and an application program for at least one crop protection product (“Spray Plan”). By actuating the respective virtual button, the user may be able to start a process for specifying a market (“Select Market”) or a process for specifying an application program for at least one crop protection product (“Create Spray Plan”).

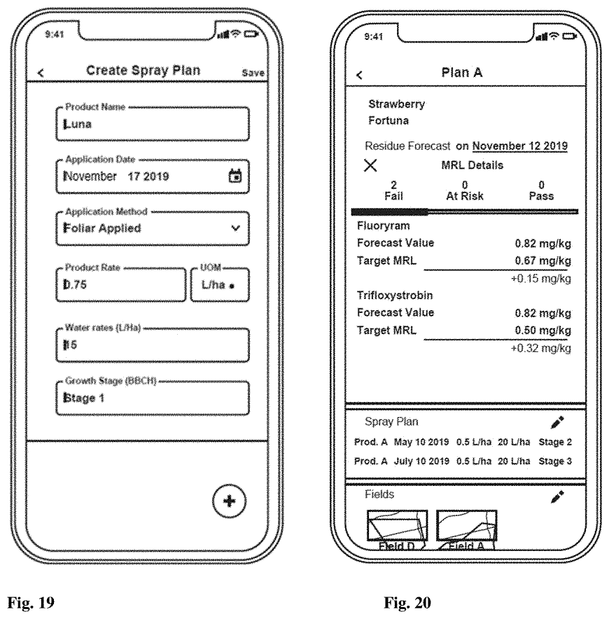

The process for specifying a market is shown in the form of images in FIG. 18. The process for specifying an application program for at least one crop protection product is shown in the form of images in FIG. 19.

The expression “market” represents a combination of a trader (or a store chain) and a country. FIG. 18 shows an overview of markets ordered by country (“Market by Country”). In the present case, store chains are listed for three countries: for Germany the store chains ALDI, Schwarz-Lidl and Rossmann, for Poland the store chains Lidl and Fresh market, and for Spain the store chains Mercadona, Lidl and ALDI. The user can select one or more store chains by clicking. In the present case, the user has selected the following combinations: Germany: ALDI, Germany: Schwarz-Lidl, Poland: Fresh market, Spain: Mercadona. By actuating the virtual button, the user is able to create further markets (“Create Custom Market”).

FIG. 19 shows a display with a number of input fields for specifying an application program for at least one crop protection product. A first input field can be used to specify the crop protection product by inputting a product name In the present case, the product name “Luna” has been input. In a further input field, the user can input and/or select the date of application of the crop protection product. In the present case, the user has specified 17 Nov. 2019 as the date. In a further input field, the user can input the application method. In the present case, the user has input leaf application (“Foliar Applied”) as the application method. In two further input fields, the user can input the amount of crop protection product (to be) deployed (“Product Rate”, “UOM” (=Unit of Measure)). In the present case, the user has input an amount of 0.75 l/ha. In a further input field, the user can input an amount of water deployed (“Water rates (L/ha)”). In the present case, the user has input an amount of 15 l/ha. In a further input field, the user can input the state of development of the crop plant (on deployment of the crop protection product) in the form of the BBCH code (“Growth Stage (BBCH)”). In the present case, the user has input BBCH code 1 (“Stage 1”).

FIG. 20 shows a display with results of a prediction. The results are predicted for the strawberry crop plant of the “Fortuna” variety. The results are based on an application program (“Plan A”, “Spray Plan”) in which 0.5 l/ha of a crop protection product designated “Prod. A” has been applied together with 20 l/ha of water at each of two junctures (10 May 2019 and 10 Jul. 2019). On 10 May 2019, the crop plant is/was at the development stage with BBCH code 2 (“Stage 2”); on 10 Jul. 2019, the crop plant is/was at the development stage with BBCH code 3 (“Stage 3”).

The crop protection product designated “Prod. A” comprises two active ingredients (fluopyram, trifloxystrobin). The date specified for which the prediction is made is 12 Nov. 2019 (“Residue Forecast on Nov. 12 2019”). The calculated (predicted) amount of fluopyram residue is 0.82 mg/kg. The calculated (predicted) amount of trifloxystrobin residue is 0.82 mg/kg. Both amounts of residue are above the MRL value (0.67 mg/kg for fluopyram, 0.5 mg/kg for trifloxystrobin).

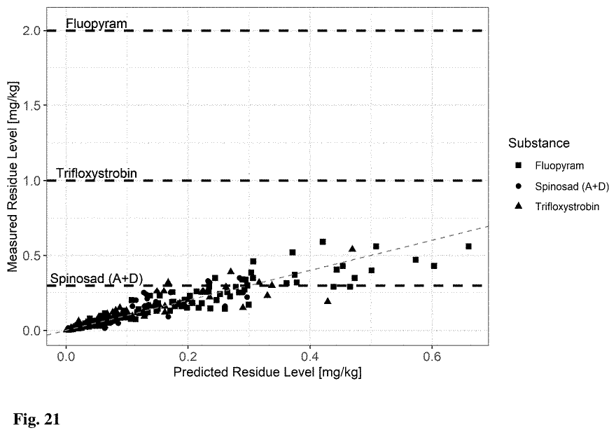

FIG. 21 shows, in the form of a graph, a comparison between measured amounts of residue (y axis (ordinate) “Measured Residues Level [mg/kg]”) and amounts of residue calculated (predicted) in accordance with the invention (x axis (abscissa) “Predicted Residue Level [mg/kg]”) for three crop protection products (fluopyram, spinosad (A+D) and trifloxystrobin). Each measurement point on the graph represents a measured amount of residue and one calculated in accordance with the invention. The measurements and calculations were ascertained for strawberries that were grown under various conditions. The prediction was made with the aid of a non-linear regression model with the following input parameters:
crop protection product used
number of applications of the crop protection product and amounts applied
period of time between the last application and the harvesting time
average air temperature on the respective days of application
average air humidity on the respective days of application
accumulated radiation energy (insolation or artificial lighting) on the respective days of application
average diameter and average height of the crop plants (strawberries) on the respective application days.

In FIG. 21, a clear correlation is seen between amounts of residue predicted and measured. Maximum levels for the crop protection product are shown as horizontal lines in the graph. The maximum levels for fluopyram and trifloxystrobin are not exceeded in the graph shown; the maximum amount for spinosad (A+D) is exceeded in some cases.

1. A device for predicting a residue of a crop protection product, the device comprising one or more processors configured to:
receive and/or ascertain input information, wherein the input information comprises
information relating to a crop plant being grown,
information relating to a growing of the crop plant,
information relating to a crop protection product that has been or will be used in the growing of the crop plant, and
information relating to an application of the crop protection product;
calculate an amount of a residue of the crop protection product in and/or on the parts of the crop plant intended for human and/or animal consumption, preferably at the time of harvesting of the crop plant, using the input information;
output information relating to the amount of the residue.

2. The device of claim 1, wherein one or more processors are further configured to:
ascertain a maximum amount of a residue of the crop protection product in the crop plant or parts thereof;
compare the calculated amount of the residue with the maximum amount ascertained; and
output information as to whether and/or to what extent the calculated amount of the residue is above or below the maximum amount.

3. The device of claim 2, wherein the maximum amount is an upper limit for a residue of the crop protection product in the crop plant or parts thereof that has been officially approved for a country or a region.

4. The device of claim 2, wherein the maximum amount is an upper limit for a residue in the crop plant or parts thereof that has been stipulated by a dealer for the crop protection product.

5. The device of claim 1, wherein the one or more processors are further configured to:
identify countries and/or regions for which the calculated amount of the residue does not exceed any officially approved upper limit for the residue in the crop plant or parts thereof and/or identify traders for which the calculated amount of residue does not exceed any stipulated upper limit for the residue in the crop plant or parts thereof; and
output information related to the identified countries and/or regions and/or traders.

6. The device of claim 1, wherein the input information includes the following information: crop plant variety being grown and/or a country and/or a region in which the crop plant is being grown, and/or an application rate of the crop protection product, and/or a length of a period of time between a juncture of application of the crop protection product and the juncture of harvesting.

7. The device of claim 1, wherein the amount of the residue of the crop protection product is calculated using at least the following input information:
the crop plant being grown;
a country or region in which the crop plant is being grown;
the crop protection product that has been or will be used;
an application rate of the crop protection product that has been or will be used; and
a length of the period of time between the juncture of application and the juncture of harvesting.

8. The device of claim 1, wherein the amount of the residue of the crop protection product is calculated using at least the following input information:
the crop plant being grown;
the crop protection product that has been or will be used;
an application rate of the crop protection product that has been or will be used;
an amount of biomass and/or fruit mass present in the crop plant being grown at the juncture of application of the crop protection product; and
a length of the period of time between the juncture of application and the juncture of harvesting.

9. The device of claim 1, wherein the amount of the residue of the crop protection product is calculated by a regression model.

10. The device of claim 1, wherein the amount of the residue of the crop protection product is calculated by a model that has been trained with empirical data using a self-learning algorithm configured to predict the amount of the residue of the crop protection product using information comprising:
the crop plant being grown;
a country or region in which the crop plant is being grown or an amount of biomass and/or fruit mass present in the crop plant being grown at the juncture of application of the crop protection product;
the crop protection product that has been or will be used;
an application rate of the crop protection product that has been or will be used; and
a length of the period of time between the juncture of application and the juncture of harvesting.

11. The device of claim 1, wherein the amount of the residue is displayed to a user as a proportion of a maximum amount stipulated by an official body and/or a trader.

12. The device of claim 1, wherein the input information is received and/or ascertained in the form of values of parameters and wherein the one or more processors are further configured to:
receive and/or ascertain further input information, wherein the further input information specifies which of the parameters are variable and which of the parameters are invariable;
vary the values of the variable parameters to ascertain values for which the amount of residue calculated reaches a minimum; and
output the values for which the amount of the residue calculated reaches a minimum.

13. A method for predicting a residue of a crop protection product, the method comprising:
receiving and/or ascertaining, by one or more processors of a computer system, input information, wherein the input information includes:
information relating to a crop plant being grown;
information relating to a growing of the crop plant;
information relating to a crop protection product that has been or will be used in the growing of the crop plant; and
information relating to an application of the crop protection product;
calculating, using the one or more processors, an amount of a residue of the crop protection product in and/or on the parts of the crop plant intended for human and/or animal consumption, preferably at the time of harvesting;
outputting, by the one or more processors, information relating to the amount of residue.

14. A system for predicting a residue of a crop protection product, the system comprising:
a first computer system comprising a first processor; and
a second computer system comprising a second processor;
wherein the first processor of the first computer system is configured to:
receive and/or ascertain the following input information comprising:
information relating to a crop plant being grown,
information relating to a growing of the crop plant,
information relating to a crop protection product that has been or will be used in the growing of the crop plant, and
information relating to an application of the crop protection product; and
transmit the input information via a network to the second computer system;
wherein the second processor of the second computer system is configured to:
receive the input information via the network;
calculate, based on the input information, an amount of a residue of the crop protection product in and/or on the parts of the crop plant intended for human and/or animal consumption, preferably at the time of harvesting; and
transmit the amount of the residue via the network to the first computer system; and
wherein the first processor is configured to receive the amount of the residue via the network, and
output the amount of the residue to a user.

15. A non-transitory computer readable storage medium storing one or more programs for predicting a residue of a crop protection product, the programs for execution by one or more processors of an electronic device, wherein, when executed, the programs cause the electronic device to:
receive and/or ascertain input information comprising:
information relating to a crop plant being grown,
information relating to a growing of the crop plant,
information relating to a crop protection product that has been or will be used in the growing of the crop plant, and
information relating to an application of the crop protection product;
calculate, using the input information, an amount of a residue of the crop protection product in and/or on the parts of the crop plant intended for human and/or animal consumption, preferably at the time of harvesting;
output information relating to the amount of the residue.
